(19) United States
(12) Patent Application Publication
(10) Pub. No.: US 2008/0038228 A1
(43) Pub. Date: Feb. 14, 2008

(54) Method

(75) Inventors: Nina Rautonen, Espoo (FI); Jussi Nurmi, Parainen (FI); Paivi Nurminen, Siuntio (FI); Pauli Puolakkainen, Espoo (FI)

Correspondence Address: STEPTOE & JOHNSON LLP, 1330 CONNECTICUT AVENUE, N.W., WASHINGTON, DC, 20036, US

(73) Assignee: Danisco A/S, Copenhagen K (DK)

(21) Appl. No.: 10/571,992

(22) PCT Filed: Sep. 24, 2004
(86) PCT No.: PCT/IB04/03273

§ 371 Date: Feb. 7, 2007

(30) Foreign Application Priority Data
Oct. 1, 2003 (GB) 0323039.8

(51) Int. Cl. A61K 35/74; A61K 31/205; A61P 29/00; A61K 35/66

(52) U.S. Cl. 424/93.4; 424/93.1

(57) ABSTRACT

The present invention relates to the use of a microorganism and/or a metabolite thereof to at least increase the amount of a COX-1 mRNA in a cell. The present invention further relates to the use of a microorganism and/or a metabolite thereof in the manufacture of a medicament to treat the side effects associated with nonsteroidal antiinflammatory drugs (NSAIDs). The present invention further relates to a pharmaceutical preparation comprising in combination a nonsteroidal antiinflammatory drug (NSAIDs) and a microorganism and/or a metabolite thereof which is capable of at least increasing the amount of a COX-1 mRNA in a cell. The present invention yet further relates to a pharmaceutical preparation comprising in combination betaine or a pharmaceutically acceptable salt thereof and a microorganism and/or a metabolite thereof which is capable of at least increasing the amount of a COX-1 mRNA in a cell. The microorganism may suitably be a bacterium, preferably from the genus Bifidobacterium.

### FIELD OF INVENTION

The present invention relates to the use of a microorganism and/or a metabolite thereof to at least increase the amount of a COX-1 mRNA in a cell.

The present invention relates to the use of a microorganism and/or a metabolite thereof in the manufacture of a medicament to treat inter alia the side effects associated with nonsteroidal anti-inflammatory drugs (NSAIDs).

The present invention further relates to a pharmaceutical preparation comprising in combination a nonsteroidal anti-inflammatory drug (NSAIDs) and a microorganism and/or a metabolite thereof which is capable of at least increasing the amount of a COX-1 mRNA in a cell.

The present invention yet further relates to the use in combination of i) a microorganism and/or a metabolite thereof and ii) betaine and/or a pharmaceutically acceptable salt thereof in the manufacture of a medicament to at least increase the amount of COX-1 mRNA in a cell.

The present invention yet further relates to the use in combination of i) a microorganism and/or a metabolite thereof and ii) betaine and/or a pharmaceutically acceptable salt thereof in the manufacture of a medicament to treat inter alia the side effects associated with nonsteroidal anti-inflammatory drugs (NSAIDs).

The present invention further relates to a pharmaceutical preparation comprising in combination i) a nonsteroidal anti-inflammatory drug (NSAIDs) and/or ii) betaine and/or a pharmaceutically acceptable salt thereof, and iii) a microorganism and/or a metabolite thereof which is capable of at least increasing the amount of a COX-1 mRNA in a cell.

### TECHNICAL BACKGROUND

Cyclooxygenase (COX), also known as prostaglandin-endoperoxide synthase (PTGS), fatty acid COX, PGH synthase and EC 1.14.99.1, is a key regulatory enzyme in the synthetic pathway of eicosanoid (i.e. prostaglandin [PG]) production. Eicosanoids are responsible for multiple inflammatory, mitogenic, and angiogenic activities in various tissue and organ systems. Therefore, COX is implicated in the production of fever, inflammation and pain.

Regardless of the etiology, a deficiency of COX results in both beneficial and detrimental physiological conditions relative to imbalances of the eicosanoids, in particular prostaglandins (PGs). The prostaglandins are a family of related compounds that contained 20-carbon polyunsaturated fatty acids with a cyclopentane ring.

There are two COX isoforms: COX-1 (PTGS-1) and COX-2 (PTGS-2) respectively.

In the literature, COX-1 is considered as a constitutive enzyme expressed in many tissues including the intestine and colon, whereas COX-2 is considered as an inducible enzyme expressed in macrophages, fibroblasts and other cell types in inflammation. COX-2 expression is induced, for example, by phorbol esters and proinflammatory cytokines, including interleukin (IL)-1β and tumour necrosis factor (TNF)-α. Infection of intestinal epithelial cells with invasive bacteria such as Salmonella also induces COX-2 expression. COX-1 is thought to be responsible for the production of the PGs associated with the maintenance of gastrointestinal integrity, whereas COX-2 is believed to be responsible for the production of PGs associated with the mediation of inflammation. Singer et al Gastroenterology 1998: 115 p 297-306 investigated the expression and cellular distribution of COX-1 and COX-2 in normal ileum, normal colon, ulcerative colitis, Crohn's colitis and Crohn's ileitis.

Numerous drugs have been identified which inhibit the COX enzyme and which have been shown to delay or prevent certain forms of cancer. These agents, known as nonsteroidal anti-inflammatory drugs (NSAIDs), occupy an important niche in clinical practise. They are used to suppress inflammation, relieve pain and fever and prevent thrombosis. Most NSAIDs are dispensed via prescription; however, some NSAIDs, including aspirin (acetylsalicylic acid), ibuprofen and naproxen, are readily available as over-the-counter medications. The use of NSAID therapy has been documented in colon cancer where it is suggested that NSAIDs block colon carginogenesis at an early stage (Prescott & Fitzpatrick Biochemica et Biophysica Acta; 1470 (2000) M69-M78). Studies of NSAID therapy have implicated the COX-2 isoenzyme as the culprit. COX-2, and its isoform COX-1, are the established pharmacological targets of NSAIDs.

As mentioned above, it is thought that COX-1 synthesises PGs that serve noble purposes in housekeeping, such as protecting the stomach from ulcers and regulating renal blood flow. COX-2 on the other hand, synthesises the excessive amounts of PGs associated with pain and fever. Conventional NSAIDs taken orally inhibit both COX-1 and COX-2 non-selectively at concentrations encountered in the gastrointestinal tract. Thus, any of the beneficial effects on the carcinogenesis, or other pathological processes, are inseparable from the risk for adverse effects in the gastrointestinal tract.

### SUMMARY ASPECTS

A seminal finding of the present invention is that the microorganisms and/or a metabolite thereof according to the present invention can be used to enhance COX-1 gene expression in a cell of a subject. In particular, a seminal finding of the present invention is that the microorganisms and/or a metabolite thereof according to the present invention can be used to increase the amount of COX-1 mRNA in a cell of a subject.

The inventors have now discovered that low COX-1 activity in a cell has a direct and/or indirect influence on the health of that cell. In particular, it has now been discovered that diseased and/or unhealthy cells (and thus tissues), contrary to the prior art teachings, in fact typically have a decreased COX-1 gene expression and/or a reduced amount of COX-1 mRNA therein compared with comparative healthy cells (and thus tissues).

Thus, the present invention is predicated upon the finding that a subject's health can be promoted by enhancing COX-1 gene expression and/or the amount of COX-1 mRNA in a cell of said subject. In particular, a subject's intestinal health can be improved by increasing COX-1 gene expression in intestinal epithelial cells, for example.

In addition, the present invention is predicated upon the surprising finding that a microorganism and/or a metabolite thereof can be used to modify the expression pattern of one or more cyclooxygenases. In particular, it has been found that a microorganism can be used to modify the amount of more than one COX mRNAs. In particular, it has been found that a microorganism can be used to enhance the expression of the COX-1 gene in a cell, and thus can be used to increase the amount of COX-1 mRNA in a cell.

The present invention is also predicated upon the surprising finding that a microorganism and/or a metabolite thereof may be used to overcome the side effects associated with the administration of nonsteroidal anti-inflammatory drugs (NSAIDs).

Furthermore, the present invention is predicated upon the surprising finding that a microorganism and/or a metabolite thereof when used in combination with betaine or a pharmaceutically acceptable salt thereof results in synergistic health benefits.

### DETAILED ASPECTS

In one aspect, the present invention provides the use of a microorganism and/or a metabolite thereof in the manufacture of a medicament for use in increasing the amount of a COX-1 mRNA in a cell.

In another aspect, the present invention provides the use of a microorganism and/or a metabolite thereof in the manufacture of a medicament for use in modifying the amount of more than one cyclooxygenase mRNA in a cell.

In a further aspect, the present invention provides the use of a microorganism and/or a metabolite thereof in the manufacture of a medicament for use in increasing the amount of a COX-1 mRNA in a cell, whilst simultaneously decreasing the amount of a COX-2 mRNA in a cell.

In another aspect, the present invention provides the use of a microorganism and/or a metabolite thereof in the manufacture of a medicament for use in promoting the health of a subject, and in particular for use in promoting the intestinal health of a subject.

In another aspect, the present invention provides the use of a microorganism and/or a metabolite thereof in the manufacture of a medicament for use in the prevention and/or treatment of one or more of the following: a dermatological disorder or disease; cancers of the gastrointestinal tract; inflammatory intestinal problems and diseases; trauma of intestinal mucosa; enteropathies; recovery from surgery and skin wounds; diarrhea; nephropathies; arteriosclerosis; hypertension; liver damage; autoimmune diseases; aging; fatigue; glomerulonephritis; infectious diseases caused by pathogenic microorganisms; alopecia areata, conjunctivitis; keratitis; gastric ulcers; ischemic bowel disease; necrotizing enterocolitis; intestinal lesions; Coeliac diseases; proctitis; anemia; sarcoidosis; fibroid lung; idiopathic interstitial pneumonia; chronic rheumatoid arthritis; multiple sclerosis; Alzheimer's disease; anorexia; migraine, arthritis deformans; asthma; hay fever; periodontal diseases; urogenital diseases, respiratory disorders and endotoxic shock.

In another aspect, the present invention provides the use of a microorganism and/or a metabolite thereof in the manufacture of a medicament for use in increasing the tolerance of a subject to immunomodulating agents and/or anti-inflammatory drugs and/or increasing the tolerance of a subject to antibiotic agents.

In one aspect the present invention provides the use of a microorganism and/or a metabolite thereof in the manufacture of a medicament for use in the prevention and/or treatment of sunburn.

In a further aspect the present invention provides the use of a microorganism and/or a metabolite thereof in the manufacture of a medicament for use in the prevention and/or treatment of reduced weight gain in livestock, preferably poultry, preferably chickens.

In a further aspect the present invention provides the use of a microorganism and/or a metabolite thereof in the manufacture of a medicament for use in the prevention and/or treatment of a side effect associated with nonsteroidal anti-inflammatory drugs (NSAIDs).

In a further aspect the present invention provides a pharmaceutical preparation comprising in combination a nonsteroidal anti-inflammatory drug (NSAID) and a microorganism and/or a metabolite thereof which is capable of at least increasing the amount of a COX-1 mRNA in a cell.

In another aspect the present invention provides a pharmaceutical preparation comprising in combination a nonsteroidal anti-inflammatory drug (NSAID) and a microorganism and/or metabolite thereof which is capable of at least increasing the amount of a COX-1 mRNA in a cell for use as a medicament.

In another aspect the present invention provides a pharmaceutical preparation comprising in combination a nonsteroidal anti-inflammatory drug (NSAID) or a pharmaceutically acceptable salt thereof and a microorganism and/or metabolite thereof which is capable of at least increasing the amount of a COX-1 mRNA in a cell admixed with a pharmaceutically acceptable carrier, diluent or excipient.

In a further aspect the present invention provides a pharmaceutical preparation comprising in combination i) betaine and/or a pharmaceutically acceptable salt thereof and ii) a microorganism and/or a metabolite thereof which is capable of at least increasing the amount of a COX-1 mRNA in a cell.

In one aspect, the present invention provides the use of a microorganism and/or a metabolite thereof to at least increase the amount of a COX-1 mRNA in a cell.

In one aspect, the present invention provides the use of a microorganism and/or a metabolite thereof to increase the amount of a COX-1 mRNA in a cell.

In another aspect, the present invention provides the use of a microorganism and/or a metabolite thereof to modify the amount of more than one cyclooxygenase mRNA in a cell.

In a further aspect, the present invention provides the use of a microorganism and/or a metabolite thereof to increase the amount of a COX-1 mRNA in a cell, whilst simultaneously decreasing the amount of a COX-2 mRNA in a cell.

The present invention further relates to a medicament comprising a microorganism and/or a metabolite thereof according to the present invention.

In one aspect, the present invention provides the use of a microorganism and/or a metabolite thereof according to the present invention to at least increase the amount of a COX-1 mRNA in a cell.

In another aspect, the present invention provides the use of a microorganism and/or a metabolite thereof according to the present invention in the manufacture of a medicament for the treatment of one or more of the following disorders, diseases or conditions: a dermatological disorder or disease; cancers of the gastrointestinal tract; inflammatory intestinal problems and diseases; trauma of intestinal mucosa; enteropathies; recovery from surgery and skin wounds; diarrhoea; nephropathies; arteriosclerosis; hypertension; liver damage; autoimmune diseases; aging; fatigue; glomerulonephritis; infectious diseases caused by pathogenic microorganisms; alopecia areata; conjunctivitis; keratitis; gastric ulcers; ischemic bowel disease; necrotizing enterocolitis; intestinal lesions; Coeliac diseases; proctitis; anemia; sarcoidosis; fibroid lung; idiopathic interstitial pneumonia; chronic rheumatoid arthritis; multiple sclerosis; Alzheimer's disease; anorexia; migraine, arthritis deformans; asthma; hay fever; periodontal diseases; urogenital diseases; respiratory disorders and endotoxic shock.

In another aspect the present invention provides the use of a microorganism and/or a metabolite thereof according to the present invention in the manufacture of a medicament to prevent and/or treat reduced weight gain in livestock, preferably poultry, preferably chickens.

In another aspect the present invention provides the use of a microorganism and/or a metabolite thereof according to the present invention in the manufacture of a medicament to increase the tolerance of a subject to immunomodulating agents and/or anti-inflammatory drugs and/or increasing the tolerance of a subject to antibiotic agents.

In one aspect, the present invention provides a method for promoting the health, preferably intestinal health of a subject, with method comprises at least increasing the amount of a COX-1 mRNA in a cell of said subject.

In a further aspect, the present invention provides a method of treating a disorder, disease or condition in a subject in need of treatment, which method comprises administering to said subject an effective amount of a microorganism and/or a metabolite thereof according to the present invention.

Suitably, the disorder, disease or condition may be one or more of the following: a dermatological disorder or disease; cancers of the gastrointestinal tract; inflammatory intestinal problems and diseases; trauma of intestinal mucosa; enteropathies; recovery from surgery and skin wounds; diarrhea; nephropathies; arteriosclerosis; hypertension; liver damage; autoimmune diseases; aging; fatigue; glomerulonephritis; infectious diseases caused by pathogenic microorganisms; alopecia areata; conjunctivitis; keratitis; gastric ulcers; ischemic bowel disease; necrotizing enterocolitis; intestinal lesions; Coeliac diseases; proctitis; anemia; sarcoidosis; fibroid lung; idiopathic interstitial pneumonia; chronic rheumatoid arthritis; multiple sclerosis; Alzheimer's disease; anorexia; migraine, arthritis deformans; asthma; hay fever; periodontal diseases; urogenital diseases; respiratory disorders and endotoxic shock.

In a further aspect, the present invention provides a method of preventing and/or treating of reduced weight gain in livestock, preferably poultry, preferably chickens, which method comprises administering to said subject an effective amount of a microorganism and/or a metabolite thereof according to the present invention.

In another aspect, the present invention provides a method of improving the health of a subject, in particular the intestinal health of a subject, which method comprises administering to said subject an effective amount of a microorganism and/or a metabolite thereof according to the present invention.

In another aspect, the present invention provides a method of improving the health of a subject, in particular the intestinal health of a subject, which method comprises administering to said subject an effective amount of a microorganism and/or a metabolite thereof, which microorganism and/or a metabolite thereof at least increases the amount of a COX-1 mRNA in at least one cell of the subject.

In a further aspect, the present invention provides a method of improving the health of a subject, in particular the intestinal health of a subject, which method comprises administering to said subject an effective amount of a microorganism and/or a metabolite thereof, which microorganism and/or a metabolite thereof modifies the amount of more than one cyclooxygenase mRNA in a cell.

In another aspect, the present invention provides a method of improving the health of a subject, in particular the intestinal health of a subject, which method comprises administering to said subject an effective amount of a microorganism and/or a metabolite thereof, which microorganism and/or a metabolite thereof increases the amount of a COX-1 mRNA in a cell, whilst simultaneously decreases the amount of a COX-2 mRNA in a cell.

In a further aspect, the present invention provides a method of preventing and/or treating of reduced weight gain in livestock, preferably poultry, preferably chickens, which method comprises administering to said subject an effective amount of a bacterium from the genus Bifidobacterium, for example Bifidobacterium species 420, and/or a metabolite thereof.

In another aspect, the present invention provides a method of preventing and/or treating one or more of a dermatological disorder or disease; cancers of the gastrointestinal tract; inflammatory intestinal problems and diseases; trauma of intestinal mucosa; enteropathies; recovery from surgery and skin wounds; diarrhoea; nephropathies; arteriosclerosis; hypertension; liver damage; autoimmune diseases; aging; fatigue; glomerulonephritis; infectious diseases caused by pathogenic microorganisms; alopecia areata; conjunctivitis; keratitis; gastric ulcers; ischemic bowel disease; necrotizing enterocolitis; intestinal lesions; Coeliac diseases; proctitis; anemia; sarcoidosis; fibroid lung; idiopathic interstitial pneumonia; chronic rheumatoid arthritis; multiple sclerosis; Alzheimer's disease; anorexia; migraine, arthritis deformans; asthma, hay fever, periodontal diseases; urogenital diseases; respiratory disorders and endotoxic shock in a subject, which method comprises administering to said subject an effective amount of a microorganism and/or a metabolite thereof, which microorganism and/or a metabolite thereof at least increases the amount of a COX-1 mRNA in at least one cell of the subject.

In a further aspect, the present invention provides a method of preventing and/or treating one or more of a dermatological disorder or disease; cancers of the gastrointestinal tract; inflammatory intestinal problems and diseases; trauma of intestinal mucosa; enteropathies; recovery from surgery and skin wounds; diarrhoea; nephropathies; arteriosclerosis; hypertension; liver damage; autoimmune diseases; aging; fatigue; glomerulonephritis; infectious diseases caused by pathogenic microorganisms; alopecia areata; conjunctivitis; keratitis; gastric ulcers; ischemic bowel disease; necrotizing enterocolitis; intestinal lesions; Coeliac diseases; proctitis; anemia; sarcoidosis; fibroid lung; idiopathic interstitial pneumonia; chronic rheumatoid arthritis; multiple sclerosis; Alzheimer's disease; anorexia; migraine, arthritis deformans; asthma; hay fever; periodontal diseases; urogenital diseases; respiratory disorders and endotoxic shock in a subject, which method comprises administering to said subject an effective amount of a microorganism and/or a metabolite thereof, which microorganism and/or a metabolite thereof modifies the amount of more than one cyclooxygenase mRNA in a cell.

In another aspect, the present invention provides a method of preventing and/or treating one or more of a dermatological disorder or disease; cancers of the gastrointestinal tract; inflammatory intestinal problems and diseases; trauma of intestinal mucosa; enteropathies; recovery from surgery and skin wounds; diarrhoea; nephropathies; arteriosclerosis; hypertension; liver damage; autoimmune diseases; aging; fatigue; glomerulonephritis; infectious diseases caused by pathogenic microorganisms; alopecia areata; conjunctivitis; keratitis; gastric ulcers; ischemic bowel disease; necrotizing enterocolitis; intestinal lesions; Coeliac diseases; proctitis; anemia; sarcoidosis; fibroid lung; idiopathic interstitial pneumonia; chronic rheumatoid arthritis; multiple sclerosis; Alzheimer's disease; anorexia; migraine, arthritis deformans; asthma; hay fever; periodontal diseases; urogenital diseases; respiratory disorders and endotoxic shock in a subject, which method comprises administering to said subject an effective amount of a microorganism and/or a metabolite thereof, which microorganism and/or a metabolite thereof increases the amount of a COX-1 mRNA in a cell, whilst simultaneously decreases the amount of a COX-2 mRNA in a cell.

In another aspect, the present invention provides a method of preventing and/or treating one or more of a dermatological disorder or disease; cancers of the gastrointestinal tract; inflammatory intestinal problems and diseases; trauma of intestinal mucosa; enteropathies; recovery from surgery and skin wounds; diarrhoea; nephropathies; arteriosclerosis; hypertension; liver damage; autoimmune diseases; aging; fatigue; glomerulonephritis; infectious diseases caused by pathogenic microorganisms; alopecia areata; conjunctivitis; keratitis; gastric ulcers; ischemic bowel disease; necrotizing enterocolitis; intestinal lesions; Coeliac diseases; proctitis; anemia; sarcoidosis; fibroid lung; idiopathic interstitial pneumonia; chronic rheumatoid arthritis; multiple sclerosis; Alzheimer's disease; anorexia; migraine, arthritis deformans; asthma; hay fever; periodontal diseases; urogenital diseases; respiratory disorders and endotoxic shock in a subject, which method comprises administering to said subject an effective amount of a bacterium from the Bifidobacterium genus, for example Bifidobacterium sp. 420, and/or a metabolite thereof.

In another aspect, the present invention provides a method of increasing the tolerance of a subject to immunomodulating agents and/or anti-inflammatory drugs particularly nonsteroidal anti-inflammatory drugs [NSAIDs]) and/or increasing the tolerance of a subject to antibiotic agents, which method comprises administering to said subject an effective amount of a microorganism and/or a metabolite thereof according to the present invention.

In another aspect, the present invention provides a method of increasing the tolerance of a subject to immunomodulating agents and/or anti-inflammatory drugs particularly nonsteroidal anti-inflammatory drugs [NSAIDs]) and/or increasing the tolerance of a subject to antibiotic agents, which method comprises administering to said subject an effective amount of a microorganism and/or a metabolite thereof, which microorganism and/or a metabolite thereof at least increases the amount of a COX-1 mRNA in at least one cell of the subject.

In another aspect, the present invention provides a method of increasing the tolerance of a subject to immunomodulating agents and/or anti-inflammatory drugs (particularly nonsteroidal anti-inflammatory drugs [NSAIDs]) and/or increasing the tolerance of a subject to antibiotic agents, which method comprises administering to said subject an effective amount of a microorganism and/or a metabolite thereof, which microorganism and/or a metabolite thereof modifies the amount of more than one cyclooxygenase mRNA in a cell.

In another aspect, the present invention provides a method of increasing the tolerance of a subject to immunomodulating agents and/or anti-inflammatory drugs (particularly nonsteroidal anti-inflammatory drugs [NSAIDs]) and/or increasing the tolerance of a subject to antibiotic agents, which method comprises administering to said subject an effective amount of a microorganism and/or a metabolite thereof, which microorganism and/or a metabolite thereof increases the amount of a COX-1 mRNA in a cell, whilst simultaneously decreases the amount of a COX-2 mRNA in a cell.

In a further aspect the present invention provides a method of treating and/or preventing the side-effects associated with the administration of nonsteroidal anti-inflammatory drugs (NSAIDs), which method comprises administering to the patient an effective amount of a microorganism and/or a metabolite thereof, which microorganism and/or metabolite thereof at least increases the amount of a COX-1 mRNA in at least one cell of the subject.

In a further aspect, the present invention provides a method of preventing and/or treating of reduced weight gain in livestock, preferably poultry, preferably chickens, which method comprises administering to said livestock an effective amount of a microorganism and/or a metabolite thereof, which microorganism and/or a metabolite thereof at least increases the amount of a COX-1 mRNA in at least one cell of said livestock.

In a further aspect, the present invention provides a method of preventing and/or treating of reduced weight gain in livestock, preferably poultry, preferably chickens, which method comprises administering to said livestock an effective amount of a microorganism and/or a metabolite thereof, which microorganism and/or a metabolite thereof modifies the amount of more than one cyclooxygenase mRNA in a cell of said livestock.

In a further aspect, the present invention provides a method of preventing and/or treating of reduced weight gain in livestock, preferably poultry, preferably chickens, which method comprises administering to said livestock an effective amount of a microorganism and/or a metabolite thereof, which microorganism and/or a metabolite thereof increases the amount of a COX-1 mRNA in a cell, whilst simultaneously decreases the amount of a COX-2 mRNA in a cell of said livestock.

In another aspect, the present invention relates to a process of preparation of a pharmaceutical composition, said process comprising admixing one or more microorganisms according to the present invention and/or one or more metabolites thereof with a pharmaceutically acceptable diluent, excipient or carrier.

In a further aspect, the present invention provides a pharmaceutical composition comprising a microorganism according to the present invention and/or a metabolite thereof admixed with a pharmaceutically acceptable carrier, diluent or excipient.

In another aspect the present invention provides a microorganism and/or a metabolite thereof according to the present invention for use as a medicament.

In another aspect the present invention provides a medicament comprising a microorganism and/or a metabolite thereof according to the present invention.

In a further aspect, the present invention provides a pharmaceutical pack comprising one or more compartments, wherein at least one of the compartments comprises a microorganism and/or a metabolite thereof according to the present invention.

In another aspect, the present invention provides a pharmaceutical pack comprising one or more compartments, wherein as least one compartment comprises one or more microorganism and/or metabolites thereof, which microorganism and/or metabolite thereof is capable of at least increasing the amount of a COX-1 mRNA in at least one cell of a subject, and the same or a further compartment comprises one or more nonsteroidal anti-inflammatory drugs (NSAIDs).

A process of preparation of a pharmaceutical composition said process comprising admixing one or more microorganisms and/or metabolites thereof, which microorganism and/or metabolite thereof is capable of at least increasing the amount of a COX-1 mRNA in at least one cell of a subject, with one or more nonsteroidal anti-inflammatory drugs (NSAIDs), and with a pharmaceutically acceptable diluent, excipient or carrier.

In a further aspect the present invention provides a food and/or feed comprising a microorganism and/or a metabolite thereof according to the present invention.

The present invention further provides the use of a microorganism and/or a metabolite thereof in combination with a prebiotic in the manufacture of a medicament for use in at least increasing the amount of COX-1 mRNA in a cell.

The present invention yet further provides a method of selecting a microorganism and/or a metabolite thereof capable of at least increasing the amount of a COX-1 mRNA in a target cell, comprising exposing a target cell to a microorganism and/or a metabolite thereof, determining the amount of a COX-1 mRNA in said target cell, identifying a microorganism and/or a metabolite thereof capable of at least increasing the amount of a COX-1 mRNA in said target cell.

In a further aspect, the present invention provides a method of screening microorganisms and/or a metabolite thereof to identify microorganisms and/or a metabolite thereof capable of increasing Cox-1 mRNA in a cell, comprising adding to one or more cells of cell line (preferably a human colorectal carcinoma cell line—Caco-2) a microbial suspension and determining the Cox-1 expression pattern in said one or more cells.

The present invention yet further provides a method of screening microorganisms and/or a metabolite thereof to identify microorganisms and/or a metabolite thereof capable of modifying the amount of more than one cyclooxygenase mRNA in a cell (preferably capable of increasing the amount of Cox-1 mRNA in a cell, whilst simultaneously decreasing the amount of Cox-2 mRNA in a cell) comprising adding to one or more cells of cell line (preferably a human colorectal carcinoma cell line—Caco-2) a microbial suspension and determining the Cox-1 expression pattern in said one or more cells.

### MORE DETAILED ASPECTS

The term “modify” as used herein means to increase or decrease. The term “modifying” as used herein should be construed accordingly.

In some embodiments, the cell may be an epithelial cell. For some aspects, the cell may be a gastrointestinal epithelial cell. This is particularly advantageous in the improvement of gastrointestinal health.

The term “increasing the amount of COX-1 mRNA in a cell” as used herein means as compared with the amount of COX-1 mRNA in a cell which has not been treated with a microorganism according to the present invention and/or a metabolite thereof.

The cell may also be referred to as a “target cell”, in which case it may be an unhealthy or diseased cell, such as a cancer cell and/or an inflamed cell.

In some embodiments, the cell may be a eukaryotic cell, suitably an animal cell, suitably a mammalian cell, for example a human cell.

In some embodiments, the microorganism and/or a metabolite thereof may be used in combination with betaine or a pharmaceutically acceptable salt thereof or a betaine replacement compound. Betaine, also known as trimethylglycine or TMG, is a quaternary ammonium compound that was first discovered in the juice of sugar beets (Beta vulgaris). Both betaine and betaine hydrochloride are available as a dietary supplement. Betaine is also available in both an anhydrate and monohydrate forms, either of which are suitable for use in accordance with the present invention. In addition, betaine is distributed widely in animals, plants and microorganisms, and rich dietary sources include seafood, especially marine vertebrates, wheat germ or bran and spinach.

Betaine is a type of zwitterionic compound, these are compounds having separate positive and negatively charged groups. Although not wishing to be bound by theory, betaine works by donating methyl (CH₃) groups.

In the present application it is envisaged that the betaine may be replaced by a betaine replacement compound.

In one embodiment, the betaine replacement compound may any methyl donor.

In another embodiment, the betaine replacement compound may have an overall neutral charge having charge-separated forms comprising an onium atom that does not bear any hydrogen atoms as substituents; and an anionic atom wherein the onium atom is not adjacent to the anionic atom. Preferably the onium atom is a carbonium atom.

Suitably, the betaine replacement compound may be derived from an amino acid wherein the amine group of the amino acid has been substituted to comprise a quaternary ammonium group of the formula R₁—NR₂R₃R₄,

wherein R₁ is the amino acid residue; and R₂, R₃ and R₄, are independently selected from hydrocarbyl groups.

More preferably R₂, R₃ and R₄ are independently selected from alkyl groups. Preferably R₂, R₃ and R₄ are independently selected from C1 to C5 alkyl groups. Preferably one or more of R₂, R₃ and R₄ are methyl groups. Preferably R₂, R₃ and R₄ are methyl groups.

Preferably the betaine replacement compound is a compound of the general formula X:

⁻O—C(O)—CH₂—N R₂R₃R₄

wherein R₂, R₃ and R₄, are as defined above.

By way of example only, the betaine replacement compound may be choline.

Surprisingly it has been found that when the microorganisms and/or a metabolite thereof according to the present invention is used in combination with betaine or a betaine replacement compound a synergistic effect can be obtained. In particular, the combination results in one or more of the following: a synergistic increase in the amount of a COX-1 mRNA in a cell; synergistically modifying the amount of more than one cyclooxygenase mRNA in a cell; synergistically increasing the amount of a COX-1 mRNA in a cell, whilst simultaneously decreasing the amount of a COX-2 mRNA in a cell; synergistically promoting the health of a subject, and in particular the intestinal health of a subject; synergistically increasing the tolerance of a subject to immunomodulating agents and/or anti-inflammatory drugs and/or synergistically increasing the tolerance of a subject to antibiotic agents.

In one embodiment the present invention relates to the use in combination of the microorganisms and/or a metabolite thereof according to the present invention and betaine (or a betaine replacement compound) in the manufacture of a medicament for the treatment of one or more of a dermatological disorder or disease; cancers of the gastrointestinal tract; inflammatory intestinal problems and diseases; trauma of intestinal mucosa; enteropathies; recovery from surgery and skin wounds; diarrhoea; nephropathies; arteriosclerosis; hypertension; liver damage; autoimmune diseases; ageing; fatigue; glomerulonephritis; infectious diseases caused by pathogenic microorganisms; alopecia areata; conjunctivitis; keratitis; gastric ulcers; ischemic bowel disease; necrotizing enterocolitis; intestinal lesions; Coeliac diseases; proctitis; anemia; sarcoidosis; fibroid lung; idiopathic interstitial pneumonia; chronic rheumatoid arthritis; multiple sclerosis; Alzheimer's disease; anorexia; migraine, arthritis deformans; asthma; hay fever; periodontal diseases; urogenital diseases; respiratory disorders and endotoxic shock.

Suitably, the present invention may relate to the use in combination of the microorganisms and/or a metabolite thereof according to the present invention and betaine (or a betaine replacement compound) in the manufacture of a medicament for the prevention and/or treatment of sunburn.

Suitably, the present invention may relate to the use in combination of the microorganisms and/or a metabolite thereof according to the present invention and betaine (or a betaine replacement compound) in the manufacture of a medicament for the prevention and/or of reduced weight gain in livestock, preferably poultry, preferably chickens.

Suitably, the present invention may relate to the use in combination of the microorganisms and/or a metabolite thereof according to the present invention and betaine in the manufacture of a medicament for the prevention and/or treatment of a side-effect associated with nonsteroidal anti inflammatory drugs (NSAIDs).

Synergy may be determined by treating a test subject or cell with the microorganisms and/or a metabolite thereof, and the betaine (or betaine replacement compound) separately and in combination, and comparing the effects; synergy is indicated when the combination produces a better effect than each treatment separately.

Suitably, the amount of a COX-1 mRNA in a cell following administration of the microorganism and/or a metabolite thereof according to the present invention is 10%, preferably 20%, preferably 30%, preferably 40%, preferably 50%, preferably 60%, preferably 70%, preferably 80%, preferably 90% greater than the amount of COX-1 mRNA in a cell which had not been exposed to a microorganism according to the present invention and/or a metabolite thereof.

Suitably, the amount of a COX-1 mRNA in a cell following administration of the microorganism according to the present invention and/or a metabolite thereof is more than 2-fold, suitably more than 3-fold, suitably more than 5-fold, suitably more than 10-fold, suitably more than 100-fold, suitably more than 1000-fold greater than the amount of COX-1 mRNA in a cell which had not been exposed to a microorganism according to the present invention and/or a metabolite thereof.

Suitably, cancer of the gastrointestinal tract as referred to herein may be one or more of the following: colon cancer, colorectal cancer, stomach cancer and oesophageal cancer.

Suitably, inflammatory intestinal problems and diseases as referred to herein may be one or more of the following inflammatory bowel disease (IBD), irritable bowel syndrome (IBS), weight loss associated with gastrointestinal disorders, ulcerative colitis, Crohn's disease, pouchitis, allergic colitis, eosinophilic colitis, actinic (radiation) colitis, diverticulitis, diverticulosis, hepatic encephalopathy, portal hypertension, bacterial peritonitis, gastrointestinal manifestations of Behcet's disease and of other autoimmune disorders, steatohepatitis and other chronic liver diseases.

Suitably, enteropathies as referred to herein may be one or more of an allergy and an infectious disease.

Suitably, the dermatological disorder or disease as referred to herein may be one or more of acne, psoriasis, sunburn, erythema nodosum, cryoglobulinemia, Sweet's syndrome, systemic mastocytosis, as well as urticarial, livedoid, and nodular vasculitis, skin cancers and dermatitis.

Suitably, the periodontal diseases as referred to herein may be one or more of gingivitis, periodontitis, halitosis and dental plaque.

In some aspects, the microorganism according to the present invention may be one or more of the following: a bacterium, a fungus, a yeast.

Suitably, the microorganism according to the present invention may be a bacterium.

Suitably, the microorganism according to the present invention may be a bacterium from one or more of the following genera: Lactococcus, Clostridium, Streptococcus, Pediococcus, Enterococcus, Leuconostoc, Carnobacterium, Propionibacterium, Bifidobacterium and Lactobacillus.

Suitably, the microorganism according to the present invention may be a microorganism from the genus Bifidobacterium.

Suitably, the microorganism according to the present invention may be one or more of the following microorganisms Bifidobacterium lactis, Bifidobacterium longum, Bifidobacterium breve, Bifidobacterium animalis.

Suitably, the microorganism according to the present invention may be Bifidobacterium sp. 420 microorganism.

For the avoidance of doubt, the taxonomic name for Bifidobacterium sp. 420 is Bifidobacterium lactis 420. These terms are used herein interchangeably.

It is noted that species which have been taxonomically classified as Bifidobacterium lactis are presently under review. In particular, it is thought that some species previously classified as being B. lactis may, in fact, be B. animalis. In the present invention for some aspects it is intended to cover both B. lactis species which may subsequently be taxonomically re-named as B. animalis and vice versa. In particular, Bifidobacterium sp. 420 has been classified previously as B. lactis 420. However, should this organism be reclassified as B. animalis 420 (or for that matter is reclassified as any other species of Bifidobacterium), it is intended that this organism is encompassed by the present invention.

In one embodiment, the microorganism according to the present invention may be Bifidobacterium sp. 420. Bifidobacterium sp. 420 is commercially available from Danisco A/S (Denmark).

As will be appreciated by the skilled person, the microorganism in accordance with the present invention and/or a metabolite thereof may be used in the treatment of any disease and/or disorder which occurs due to a distorted COX-1/COX-2 gene expression.

Without wishing to be bound by theory, it has been found that a microorganism according to the present invention and/or a metabolite thereof may enhance the growth of a cell, such as an epithelial cell, of a subject. For example, it has been found that the microorganism according to the present invention and/or a metabolite thereof may enhance the growth of gastrointestinal epithelial cells of a subject, such as livestock for example poultry, such as chickens for example. In which case this may promote the uptake of nutrients by the intestine of the livestock, and thus result in enhanced growth of the livestock and/or prevention of reduced weight gain of the livestock.

Suitably, the microorganism in accordance with the present invention may be in a preparation comprising at least one species of microorganism and/or a metabolite from at least one species of microorganism, suitably at least two species, suitably at least three species.

Suitably, when a metabolite of a microorganism according to the present invention is utilised, at least one metabolite may be utilised, suitably at least two metabolites may be utilised, suitably at least three metabolites may be utilised, suitably at least four metabolites may be utilised.

For some embodiments, the microorganism in accordance with the present invention may be viable.

For some embodiments, the microorganism in accordance with the present invention may be dead or non-viable.

Without wishing to be bound by theory, it is believed that the soluble metabolites associated with, for example produced by, the microorganism may be causing the advantageous effect of the microorganism. For some aspects, it is therefore unnecessary for the microorganism cells to be in direct contact with the target cells.

For some aspects, it is believed that one or more metabolites associated with, for example produced by, the microorganism may be suitable for achieving the beneficial effects taught herein. In such instances, it may be unnecessary to include the microorganisms themselves.

The term “metabolite thereof” as used herein means one or more compounds either extracted from the microorganism according to the present invention or obtained from a culture medium in which a microorganism according to the present invention is or was cultured. In some aspects the metabolite may be a crude extract of the culture medium and/or microorganism. Suitably, for some aspects the metabolite may be one or more compounds isolated and/or purified from the culture medium and/or the microorganism.

Suitably, the microorganism in accordance with the present invention may be co-cultured with one or more target cells thus allowing the transfer of soluble fractions. Suitably, the microorganism may not be in cell to cell contact with the target cell(s).

Suitably, the microorganism according to the present invention or the metabolite thereof may be freeze-dried.

Suitably the nonsteroidal anti-inflammatory drugs (NSAIDs) according to the present invention, may be one or more the following NSAIDs: aspirin (acetylsalicyl acid), ibuprofen, ketoprofen, naproxen sodium, diclofenac potassium, diclofenac sodium, etodolac, flurbiprofen, indomethacin, ketorolac, nabumetone, naproxen, oxaprozin, piroxicam and sulindac. This list is by way of example and is in no way meant to be limiting. As will be appreciated by the person skilled in the art the present invention may be considered advantageous for use with any NSAID.

The side-effects caused by NSAIDs and which may be effectively treated and/or prevented using a microorganism according to the present invention and/or a metabolite thereof may by any side-effect caused by the use of NSAIDs. Such drugs may be administered orally or topically for instance. The side effects caused by any form of administration of NSAIDs are encompassed within the present invention. By way of example only, the side-effects caused by ingesting NSAIDs may include one or more of the following: damage caused to the large and small intestine, such as specific or non-specific colitis (in particular fenemates), ulcers, bleeding, inflammatory bowel disease, small intestine perforations and strictures, and inflammation of the large or small intestine. The side effects caused by administration of NSAIDs topically include, for example, one or more of the following: exanthema, phototoxic or photoallergic dermatitis. Other side effects caused by administration of NSAIDs include stomach pain or cramps, nausea, vomiting, indigestion, diarrhoea, heart burn, headaches, drowsiness, dizziness, confusion, lightheadedness, blurred vision, rashes, skin redness, itching, blisters, skin discolourations, sensitivity to sunlight which can cause severe sunburn. The microorganism according to the present invention and/or a metabolite thereof may be used to effectively treat and/or prevent one or more of the side effects caused by NSAIDs.

### Medicament

The term “medicament” as used herein encompasses medicaments for both human and animal usage in human and veterinary medicine. In addition, the term “medicament” as used herein means any substance which provides a therapeutic and/or beneficial effect. The term “medicament” as used herein is not necessarily limited to substances which need Marketing Approval, but may include substances which can be used in cosmetics, nutraceuticals, food (including feeds and beverages for example), probiotic cultures, natural remedies. In addition, the term “medicament” as used herein encompasses a product designed for incorporation in livestock (animal) feed, particularly poultry feed.

### Treatment

It is to be appreciated that all references herein to treatment include curative, palliative and prophylactic treatment

### Substantially Pure Form and/or Isolated Form

For some aspects the microorganism and/or metabolite according to the present invention may be in a substantially pure form or is in an isolated form.

The term “substantially pure form” is used to indicate that the microorganism and/or metabolite according to the present invention is present at a high level. When the microorganism and/or metabolite is in a substantially pure form, the microorganism and/or metabolite is desirably the predominant component present in a composition. Preferably it is present at a level of more than 30%, of more than 50%, of more than 75%, of more than 90%, or even of more than 95%, said level being determined on a dry weight/dry weight basis with respect to the total composition under consideration.

At very high levels (e.g. at levels of more than 90% of more than 95% or of more than 99%) the component may be regarded as being “isolated”. Biologically active substances of the present invention (including polypeptides, nucleic acid molecules, moieties identified/identifiable via screening, etc.) may be provided in a form that is substantially free of one or more contaminants with which the substance might otherwise be associated. Thus, for example, they may be substantially free of one or more potentially contaminating polypeptides and/or nucleic acid molecules. They may be provided in a form that is substantially free of other cell components (e.g. of cell membranes, of cytoplasm, etc.). When a composition is substantially free of a given contaminant, the contaminant will be at a low level (e.g. at a level of less than 10%, less than 5% or less than 1% on the dry weight/dry weight basis set out above).

### Microorganism

Suitable viable microorganisms for use in the present invention include bacteria, moulds and/or yeasts.

Preferably, the viable microorganisms for use in the present invention are viable bacteria.

The term “viable micro-organism” means a microorganism which is metabolically active.

The microorganism may be a naturally occurring microorganism or it may be a transformed microorganism. The microorganism may also be a combination of suitable microorganisms.

Suitably, the microorganism according to the present invention may be a bacterium from one or more of the following genera: Lactococcus, Clostridium, Streptococcus, Pediococcus, Enterococcus, Leuconostoc, Carnobacterium, Propionibacterium, Bifidobacterium and Lactobacillus.

Suitably, the microorganism according to the present invention may be a microorganism from the genus Bifidobacterium.

Suitably, the microorganism according to the present invention may be one or more of the following microorganisms Bifidobacterium lactis, Bifidobacterium longum, Bifidobacterium breve, Bifidobacterium animalis.

Suitably, the microorganism according to the present invention may be Bifidobacterium sp. 420 microorganism.

For the avoidance of doubt, the taxonomic name for Bifidobacterium sp. 420 is Bifidobacterium lactis 420. These terms are used herein interchangeably.

It is noted that species which have been taxonomically classified as Bifidobacterium lactis are presently under review. In particular, it is thought that some species previously classified as being B. lactis may, in fact, be B. animalis. In the present invention for some aspects it is intended to cover both B. lactis species which may subsequently be taxonomically re-named as B. animalis and vice versa. In particular, Bifidobacterium sp. 420 has been classified previously as B. lactis 420. However, should this organism be reclassified as B. animalis 420 (or for that matter is reclassified as any other species of Bifidobacterium), it is intended that this organism is encompassed by the present invention.

In one embodiment, the microorganism according to the present invention may be Bifidobacterium sp. 420. Bifidobacterium sp. 420 is commercially available from Danisco A/S (Denmark).

Advantageously, where the product is a foodstuff, the viable micro-organism and/or soluble metabolites produced by said micro-organism should remain effective through the normal “sell-by” or “expiration” date during which the food product is offered for sale by the retailer. Preferably, the effective time should extend past such dates until the end of the normal freshness period when food spoilage becomes apparent. The desired lengths of time and normal shelf life will vary from foodstuff to foodstuff and those of ordinary skill in the art will recognise that shelf-life times will vary upon the type of foodstuff, the size of the foodstuff, storage temperatures, processing conditions, packaging material and packaging equipment.

### Caco-2 Cell-Based Exposure Assay

The human colorectal carcinoma cell line Caco-2 is grown at 37° C. and 5% CO₂ in serum-free Dulbeccos' MEM (Gibco) supplemented with 1 mM sodium pyruvate (Gibco) and 1× non-essential amino acids (Gibco) (DMEM). When grown without added microorganisms, 20 Uml⁻¹ penicillin (Gibco), 20 μgml⁻¹ streptomycin (Gibco) and 0.5 μgml⁻¹ amphotericin (Gibco) is added to the medium.

To determine the effects of various microorganisms and/or a metabolite thereof on the Cox-1/Cox-2 expression pattern, approximately 500 000 Caco-2 cells/well are seeded on 24-well cell culture plates. The cells are allowed to attach for 24 hours, after which the media is replaced by fresh media (total volume 1 ml/well) containing either no other added components than the ones described above (control treatments, with and without antibiotics) or antibiotic supplemented media with 5 mM sodium butyrate; 5 mM sodium propionate; 5 mM sodium acetate or 5 mM sodium lactate. In addition, wells are prepared that contained no antibiotics but a 0.2 μm anopore membrane tissue culture insert (Nunc, Denmark) into which 100 μl of a microbial suspension is added.

After 24 hour exposure, media and culture inserts are discarded, cells are lysed and RNA is extracted using Qiagen's (Germany) RNEasy Mini Kit. DNA is digested using the same manufacturer's RNase free DNase. Reverse transcription is performed using the High Capacity cDNA Archive Kit (Applied Biosystems, USA) according to the instructions provided by the manufacturer. Cox-1/Cox-2 expression patterns were determined by real-time quantitative TaqMan PCR (Holland et al., 1991 Proc. Natl. Acad. Sci. USA August 15; 88(16): 7276-80; and Livak and Scmittgen, 2001 Methods December; 25(4):402-8) using the default settings of an ABIPrism 7000 Sequence Detection instrument (Applied Biosystems).

The microbial suspension for use in the above assay may be prepared by culturing the microorganism on a suitable medium. The culture may be centrifuged to form a cell pellet which may be subsequently suspended in a suitable medium, e.g. DMEM.

Microorganisms which cause an increase in Cox-1 expression level and/or increase the Cox-1/Cox-2 ratio compared with an untreated control, may be microorganisms according to the present invention and/or which can be used in accordance with the present invention.

A skilled person would readily be able to screen probiotic and non-probiotic microorganisms using the “Caco-2 cell-based exposure assay” to identify specific microorganisms, additional to the ones specifically taught herein, capable of producing the claimed effect.

### Combination with Other Components

The composition of the present invention may be used in combination with other components. Thus, the present invention also relates to combinations. The microorganism and/or metabolite thereof may be referred to herein as “the composition of the present invention”.

The combination of the present invention comprises the composition of the present invention and another component which is suitable for animal or human consumption and is capable of providing a medical or physiological benefit to the consumer.

Other components of the combinations of the present invention include polydextrose, such as Litesse®, and/or a maltodextrin. These other components may be optionally added to the composition to assist the drying process and help the survival of the microorganisms.

Further examples of other suitable components include one or more of: thickeners, gelling agents, emulsifiers, binders, crystal modifiers, sweeteners (including artificial sweeteners), rheology modifiers, stabilisers, anti-oxidants, dyes, enzymes, carriers, vehicles, excipients, diluents, lubricating agents, flavouring agents, colouring matter, suspending agents, disintegrants, granulation binders etc. These other components may be natural. These other components may be prepared by use of chemical and/or enzymatic techniques.

As used herein the term “thickener or gelling agent” refers to a product that prevents separation by slowing or preventing the movement of particles, either droplets of immiscible liquids, air or insoluble solids. Thickening occurs when individual hydrated molecules cause an increase in viscosity, slowing the separation. Gelation occurs when the hydrated molecules link to form a three-dimensional network that traps the particles, thereby immobilising them.

The term “stabiliser” as used here is defined as an ingredient or combination of ingredients that keeps a product (e.g. a food product) from changing over time.

The term “emulsifier” as used herein refers to an ingredient (e.g. a food product ingredient) that prevents the separation of emulsions. Emulsions are two immiscible substances, one present in droplet form, contained within the other. Emulsions can consist of oil-in-water, where the droplet or dispersed phase is oil and the continuous phase is water; or water-in-oil, where the water becomes the dispersed phase and the continuous phase is oil. Foams, which are gas-in-liquid, and suspensions, which are solid-in-liquid, can also be stabilised through the use of emulsifiers. Aeration can occur in a three-phase system where air is entrapped by liquid oil then stabilised by agglomerated fat crystals stabilised with an emulsifier. Emulsifiers have a polar group with an affinity for water (hydrophilic) and a non-polar group which is attracted to oil (lipophilic). They are absorbed at the interfaces of the two substances, providing an interfacial film acting to stabilise the emulsion. The hydrophilic/lipophilic properties of emulsifiers are affected by the structure of the molecule. These properties are identified by the hydrophilic/lipophilic balance (HLB) value. Low HLB values indicate greater lipophilic tendencies which are used to stabilise water-in-oil emulsions. High HLB values are assigned to hydrophilic emulsifiers, typically used in oil-in-water emulsions. These values are derived from simple systems. Because foods often contain other ingredients that affect the emulsification properties, the HLB values may not always be a reliable guide for emulsifier selection.

As used herein the term “binder” refers to an ingredient (e.g. a food ingredient) that binds the product together through a physical or chemical reaction. During “elation” for instance, water is absorbed, providing a binding effect. However, binders can absorb other liquids, such as oils, holding them within the product. In the context of the present invention binders would typically be used in solid or low-moisture products for instance baking products: pastries, doughnuts, bread and others.

The term “crystal modifier” as used herein refers to an ingredient (e.g. a food ingredient) that affects the crystallisation of either fat or water. Stabilisation of ice crystals is important for two reasons. The first is directly related to the product stability from a separation standpoint The more freeze/thaw cycles a product encounters, the larger the ice crystals become. These large crystals can break down product structure, either naturally occurring, as in the case of cell walls, or that which is created by “elation”. Because the water is no longer held in place, the product may exhibit syneresis, or weeping, after thawing. Secondly, in the case of a product which is consumed frozen, these large crystals result in an undesirable, gritty mouth feel.

“Carriers” or “vehicles” mean materials suitable for compound administration and include any such material known in the art such as, for example, any liquid, gel, solvent, liquid diluent, solubilizer, or the like, which is non-toxic and which does not interact with any components of the composition in a deleterious manner.

Examples of nutritionally acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Examples of excipients include one or more of: microcrystalline cellulose and other celluloses, lactose, sodium citrate, calcium carbonate, dibasic calcium phosphate, glycine, starch, milk sugar and high molecular weight polyethylene glycols.

Examples of disintegrants include one or more of: starch (preferably corn, potato or tapioca starch), sodium starch glycollate, croscarmellose sodium and certain complex silicates.

Examples of granulation binders include one or more of: polyvinylpyrrolidone, hydroxypropylmethylcellulose (HPMC), hydroxypropylcellulose (HPC), sucrose, maltose, gelatin and acacia.

Examples of lubricating agents include one or more of: magnesium stearate, stearic acid, glyceryl behenate and talc.

Examples of diluents include one or more of: water, ethanol, propylene glycol and glycerin, and combinations thereof.

The other components may be used simultaneously (e.g. when they are in admixture together or even when they are delivered by different routes) or sequentially (e.g. they may be delivered by different routes).

Preferably, when the composition of the present invention when admixed with any other components, the microorganisms remain viable.

As used herein the term “component suitable for animal or human consumption” means a compound which is or can be added to the composition of the present invention as a supplement which may be of nutritional benefit, a fibre substitute or have a generally beneficial effect to the consumer. The ingredients can be used in a wide variety of products that require gelling, texturising, stabilising, suspending, film-forming and structuring, retention of juiciness, without adding unnecessary viscosity. Preferably, the ingredients will be able to improve the shelf life and stability of the viable culture.

The components may be prebiotics such as alginate, xanthan, pectin, locust bean gum (LBG), inulin, guar gum, galacto-oligosaccharide (GOS), fructo-oligosaccharide (FOS), polydextrose (i.e. Litesse®), lactosucrose, soybean oligosaccharides, palatinose, isomalto-oligosaccharides, gluco-oligosaccharides and xylo-oligosaccharides.

The optimum amount of the composition to be used in the combination of the present invention will depend on the product to be treated and/or the method of contacting the product with the composition and/or the intended use for the same. The amount of viable microorganism used in the compositions should be a sufficient amount to be effective and to remain sufficiently effective in improving the aroma, flavour, mildness, consistency, texture, body, mouth feel, viscosity, structure and/or organoleptic properties, nutrition and/or health benefits of food products containing said composition. This length of time for effectiveness should extend up to at least the time of utilisation of the product.

### Pharmaceutical Combinations

The microorganism of the present invention and/or a metabolite thereof may be used in combination with one or more drugs, in particular one or more drugs which cause adverse effects due to the inhibition of COX-1 gene expression. In particular, the drug may be one or more nonsteroidal anti-inflammatory drug (NSAIDs) for example.

The drug may be administered simultaneously with (e.g. when they are in admixture together or even when they are delivered by different routes) or sequentially to (e.g. when delivered by the same or different routes) the microorganism according to the present invention and/or a metabolite thereof.

In addition or alternatively, the microorganism of the present invention and/or a metabolite thereof may be used in combination with betaine or a pharmaceutically acceptable salt thereof or a betaine replacement compound for example.

The betaine or pharmaceutically acceptable salt thereof or betaine replacement compound may be administered simultaneously with (e.g. when they are in admixture together or even when they are delivered by different routes) or sequentially to (e.g. when delivered by the same or different routes) the microorganism according to the present invention and/or a metabolite thereof.

### Concentrates

The compositions for use in the present invention may be in the form of concentrates. Typically these concentrates comprise a substantially high concentration of a viable microorganism and/or a metabolite thereof. The microorganism and/or metabolite thereof may be referred to herein as “the composition of the present invention” or “compositions”.

Powders, granules and liquid compositions in the form of concentrates may be diluted with water or resuspended in water or other suitable diluents, for example, an appropriate growth medium such as milk or mineral or vegetable oils, to give compositions ready for use.

The combinations of the present invention in the form of concentrates may be prepared according to methods known in the art.

In one aspect of the present invention the product is contacted by a composition in a concentrated form. Preferably, the product is contacted by a spray-dried and/or resuspended composition.

The compositions of the present invention may be spray-dried or freeze-dried by methods known in the art.

Typical processes for making particles using a spray drying process involve a solid material which is dissolved in an appropriate solvent (e.g. a culture of a micro-organism in a fermentation medium). Alternatively, the material can be suspended or emulsified in a non-solvent to form a suspension or emulsion. Other ingredients (as discussed above) or components such as anti-microbial agents, stabilising agents, dyes and agents assisting with the drying process may optionally be added at this stage.

The solution then is atomised to form a fine mist of droplets. The droplets immediately enter a drying chamber where they contact a drying gas. The solvent is evaporated from the droplets into the drying gas to solidify the droplets, thereby forming particles. The particles are then separated from the drying gas and collected.

### Products

Any product which can benefit from the composition may be used in the present invention. These include but are not limited to fruit conserves and dairy foods and dairy food-derived products, cosmetic and pharmaceutical products. The microorganism and/or metabolite thereof may be referred to herein as “the composition of the present invention” or “the composition”.

By way of example, the composition of the present invention can be used as an ingredient to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, balanced foods and drinks, fruit fillings, care glaze, chocolate bakery filling, cheese cake flavoured filling, fruit flavoured cake filling, cake and doughnut icing, instant bakery filling creams, fillings for cookies, ready-to-use bakery filling, reduced calorie filling, adult nutritional beverage, acidified soy/juice beverage, aseptic/retorted chocolate drink, bar mixes, beverage powders, calcium fortified soy/plaim and chocolate milk, calcium fortified coffee beverage.

The composition can further be used as an ingredient in food products such as American cheese sauce, anti-caking agent for grated & shredded cheese, chip dip, cream cheese, dry blended whip topping fat free sour cream, freeze/thaw dairy whipping cream, freeze/thaw stable whipped tipping, low fat and light natural cheddar cheese, low fat Swiss style yoghurt, aerated frozen desserts, hard pack ice cream, label friendly, improved economics & indulgence of hard pack ice cream, low fat ice cream: soft serve, barbecue sauce, cheese dip sauce, cottage cheese dressing, dry mix Alfredo sauce, mix cheese sauce, dry mix tomato sauce and others.

For certain aspects, preferably the present invention may be used in connection with yoghurt production, such as fermented yoghurt drink, yoghurt, drinking yoghurt, cheese, fermented cream, milk based desserts and others.

Suitably, the composition can be further used as an ingredient in one or more of cheese applications, meat applications, or applications comprising protective cultures.

The present invention also provides a method of preparing a food or a food ingredient, the method comprising admixing the composition according to the present invention with another food ingredient.

Advantageously, the present invention relates to products that have been contacted with the composition of the present invention (and optionally with other components/ingredients), wherein the composition is used in an amount to be capable of improving the nutrition and/or health benefits of the product.

As used herein the term “contacted” refers to the indirect or direct application of the composition of the present invention to the product. Examples of the application methods which may be used, include, but are not limited to, treating the product in a material comprising the composition, direct application by mixing the composition with the product, spraying the composition onto the product surface or dipping the product into a preparation of the composition.

Where the product of the invention is a foodstuff, the composition of the present invention is preferably admixed with the product. Alternatively, the composition may be included in the emulsion or raw ingredients of a foodstuff. In a further alternative, the composition may be applied as a seasoning, glaze, colorant mixture, and the like.

For some applications, it is important that the composition is made available on or to the surface of a product to be affected/treated. This allows the composition to impart one or more of the following favourable characteristics: nutrition and/or health benefits.

The compositions of the present invention may be applied to intersperse, coat and/or impregnate a product with a controlled amount of a viable microorganism.

### Food

The composition of the present invention may be used as—or in the preparation of—a food. Here, the term “food” is used in a broad sense—and covers food for humans as well as food for animals (i.e. a feed). In a preferred aspect, the food is for human consumption.

The food may be in the form of a solution or as a solid—depending on the use and/or the mode of application and/or the mode of administration.

When used as—or in the preparation of—a food—such as functional food—the composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

Preferably, the composition is used to ferment milk or sucrose fortified milk or lactic media with sucrose and/or maltose where the resulting media containing all components of the composition—i.e. said microorganism according to the present invention—can be added as an ingredient to yoghurt milk in suitable concentrations—such as for example in concentrations in the final product which offer a daily dose of 10⁶-10¹⁰ cfu. The microorganism according to the present invention may be used before or after fermentation of the yoghurt.

For some aspects the microorganisms according to the present invention are used as—or in the preparation of—livestock feeds, in particular poultry (such as chicken) feed.

### Food Ingredient

The composition of the present invention may be used as a food ingredient and/or feed ingredient.

As used herein the term “food ingredient” or “feed ingredient” includes a formulation which is or can be added to functional foods or foodstuffs as a nutritional supplement

The food ingredient may be in the from of a solution or as a solid—depending on the use and/or the mode of application and/or the mode of administration.

### Food Supplements

The composition of the present invention may be—or may be added to—food supplements.

### Functional Foods

The composition of the present invention may be—or may be added to—functional foods.

As used herein, the term “functional food” means food which is capable of providing not only a nutritional effect, but is also capable of delivering a further beneficial effect to consumer.

Accordingly, functional foods are ordinary foods that have components or ingredients (such as those described herein) incorporated into them that impart to the food a specific functional—e.g. medical or physiological benefit—other than a purely nutritional effect.

Although there is no legal definition of a functional food, most of the parties with an interest in this area agree that they are foods marketed as having specific health effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

Surveys have suggested that consumers place the most emphasis on functional food claims relating to heart disease. Preventing cancer is another aspect of nutrition which interests consumers a great deal, but interestingly this is the area that consumers feel they can exert least control over. In fact, according to the World Health Organization, at least 35% of cancer cases are diet-related. Furthermore claims relating to osteoporosis, gut health and obesity effects are also key factors that are likely to incite functional food purchase and drive market development.

### Probiotic

For some applications, it is believed that the viable lactic acid microorganisms in the composition of the present invention can exert a probiotic culture effect. It is also within the scope of the present invention to add to the composition of the present invention further probiotic and/or prebiotics.

Here, a prebiotic is:

“a non-digestible food ingredient that beneficially affects the host by selectively stimulating the growth and/or the activity of one or a limited number of beneficial bacteria”.

The term “probiotic culture” as used herein defines live microorganisms (including bacteria or yeasts for example) which, when for example ingested or locally applied in sufficient numbers, beneficially affects the host organism, i.e. by conferring one or more demonstrable health benefits on the host organism. Probiotics may improve the microbial balance in one or more mucosal surfaces. For example, the mucosal surface may be the intestine, the urinatory tract, the respiratory tract or the skin. The term “probiotic” as used herein also encompasses live microorganisms that can stimulate the beneficial branches of the immune system and at the same time decrease the inflammatory reactions in a mucosal surface, for example the gut. In this regard, the use of the composition of the present invention, containing said probiotic ingredient for anti-cancer therapy and prevention of allergies and ulcerative colitis is also contemplated.

Whilst there are no lower or upper limits for probiotic intake, it has been suggested that at least 10⁶-10¹⁰, preferably 10⁸-10⁹, cfu as a daily dose will be effective to achieve the beneficial health effects in a host organism, such as a human.

In addition to the probiotic effect the microorganism according to the present invention may have, it is also within the scope of the present invention to provide prebiotics as other compounds which can be included in a combination along with the composition. The microorganism according to the present invention and/or a metabolite thereof may be herein referred to as “the composition”. The prebiotic component of the combination comprising the composition of the present invention are characterised with slow fermentation in the large bowel. Such prebiotics can exert a positive effect on the gut flora, specifically in the left side of the colon, an area of the gut which is especially prone to disorders in particular bowel cancer and ulcerative colitis.

Prebiotics are typically non-digestible carbohydrate (oligo- or polysaccharides) or a sugar alcohol which is not degraded or absorbed in the upper digestive tract. Known prebiotics used in commercial products and useful in accordance with the present invention include inulin (fructo-oligosaccharide, or FOS) and transgalacto-oligosaccharides (GOS or TOS). Other suitable, prebiotics include palatinoseoligosaccharide, soybean oligosaccharide, gentiooligosaccharide, xylooligomers, non-degradable starch, lactosaccharose, lactulose, lactitol, maltitol, polydextrose (i.e. Litesse®) or the like.

The prebiotic may be administered simultaneously with (e.g. in admixture together with or delivered simultaneously by the same or different routes) or sequentially to (e.g. by the same or different routes) the microorganism according to the present invention and/or a metabolite thereof.

The present invention contemplates the use of a microorganism and/and/or a metabolite thereof in combination with a prebiotic in the manufacture of a medicament for use in at least increasing the amount of COX-1 mRNA in a cell.

### Synbiotics

The present invention also contemplates using both pre- and probiotics as ingredients in a combination along with the composition of the present invention which when combined, become synbiotics. The microorganism according the present invention and/or a metabolite thereof may be referred to herein as “the composition”. The purpose of this is to combine the effects of new beneficial bacteria and the stimulation of the body-own beneficial bacteria There is a high potential in the development and the consumption of such mixtures, since some of these may well show powerful synergistic nutritional and/or health effects.

Thus the composition of the present invention may be specifically designed to contain different components which can provide a synbiotic effect to the consumer.

### Pharmaceutical

The composition of the present invention may be used as—or in the preparation of—a pharmaceutical. Here, the term “pharmaceutical” is used in a broad sense—and covers pharmaceuticals for humans as well as pharmaceuticals for animals (i.e. veterinary applications). In a preferred aspect, the pharmaceutical is for human use and/or for animal husbandry.

The pharmaceutical can be for therapeutic purposes—which may be curative or palliative or preventative in nature. The pharmaceutical may even be for diagnostic purposes.

When used as—or in the preparation of—a pharmaceutical, the composition of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

The pharmaceutical may be in the from of a solution or as a solid—depending on the use and/or the mode of application and/or the mode of administration.

### Pharmaceutical Ingredient

The microorganisms of the present invention may be used as pharmaceutical ingredients. Here, the composition may be the sole active component or it may be at least one of a number (i.e. 2 or more) of active components.

The pharmaceutical ingredient may be in the form of a solution or as a solid—depending on the use and/or the mode of application and/or the mode of administration.

### Forms

The microorganism of the present invention and/or a metabolite thereof may be used in any suitable form—whether when alone or when present in a combination with other components or ingredients. The microorganism of the present invention and/or a metabolite thereof may be referred to herein as “the composition”. Likewise, combinations comprising the composition of the present invention and other components and/or ingredients (i.e. ingredients—such as food ingredients, functional food ingredients or pharmaceutical ingredients) may be used in any suitable form.

The microorganism of the present invention may be used in the form of solid or liquid preparations or alternatives thereof. Examples of solid preparations include, but are not limited to tablets, capsules, dusts, granules and powders which may be wettable, spray-dried or freeze-dried. Examples of liquid preparations include, but are not limited to, aqueous, organic or aqueous-organic solutions, suspensions and emulsions.

Suitable examples of forms include one or more of: tablets, pills, capsules, ovules, solutions or suspensions, which may contain flavouring or colouring agents, for immediate-, delayed-, modified-, sustained-, pulsed- or controlled-release applications.

By way of example, if the composition of the present invention is used in a tablet form—such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Preferred excipients for the forms include lactose, starch, a cellulose, milk sugar or high molecular weight polyethylene glycols.

For aqueous suspensions and/or elixirs, the composition of the present invention may be combined with various sweetening or flavouring agents, colouring matter or dyes, with emulsifying and/or suspending agents and with diluents such as water, propylene glycol and glycerin, and combinations thereof.

The forms may also include gelatin capsules; fibre capsules, fibre tablets etc.; or even fibre beverages.

Further examples of form is in the form of a cream for example. For some aspects the microorganism and/or a metabolite thereof may be included in pharmaceutical and/or cosmetic creams such as sun creams and/or after-sun creams for example.

In one aspect, the composition according to the present invention may be administered in an aerosol, for example by way of a nasal spray, for instance for administration to the respiratory tract.

The invention will now be further described only by way of example in which reference is made to the following examples and Figures:

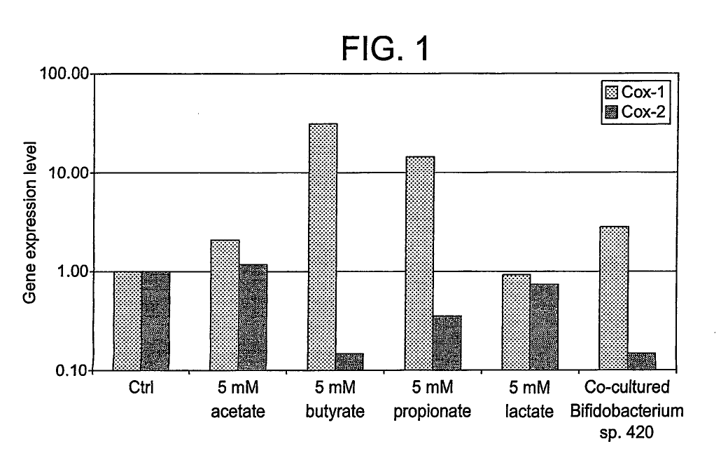

FIG. 1 shows the effect of acetate, butyrate, propionate, lactate and co-cultured Bifidobacterium sp. 420 on Cox-1/Cox-2 gene expression pattern.

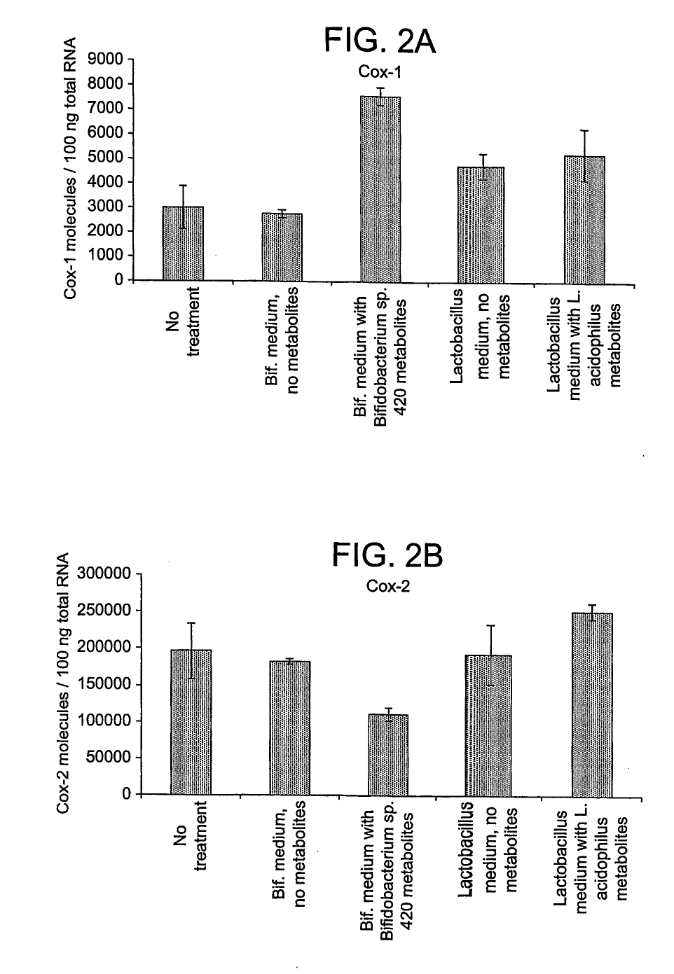

FIG. 2A shows the effects of Bifidobacterium strain 420 and L. acidophilus on Cox-1 expression. Bifidobacterium causes a significant (2.7-fold) increase; L acidophilus has no significant effect.

FIG. 2B shows the effects of Bifidobacterium strain 420 and L. acidophilus on Cox-2 expression. Bifidobacterium causes a significant (40%) increase; L acidophilus causes a significant (30%) increase.

FIG. 2C shows the effects of metabolites produced by Bifidobacterium strain 420 and L. acidophilus on Cox-1/Cox-2 ratio. Bifidobacterium causes a significant (2.7-fold) increase; L acidophilus has no significant effect.

FIG. 2D show the effects of soluble metabolites produced by five bacterial strains on intestinal epithelial Cox-1/Cox-2 expression pattern. Microbial metabolites were given as 10% filtered bacterial growth media diluted in DMEM. In the control treatment, Caco-2 cells were maintained in DMEM; in the other control treatments (fresh BIF, MRS or TSB medium), Caco-2 cells were given 10% unconditioned bacterial growth media diluted in DMEM. Bifidobacteria cause a clear increase in Cox-1/Cox-2 ratio, whereas L. acidophilus, E. coli and S. enteritidis have no significant effect.

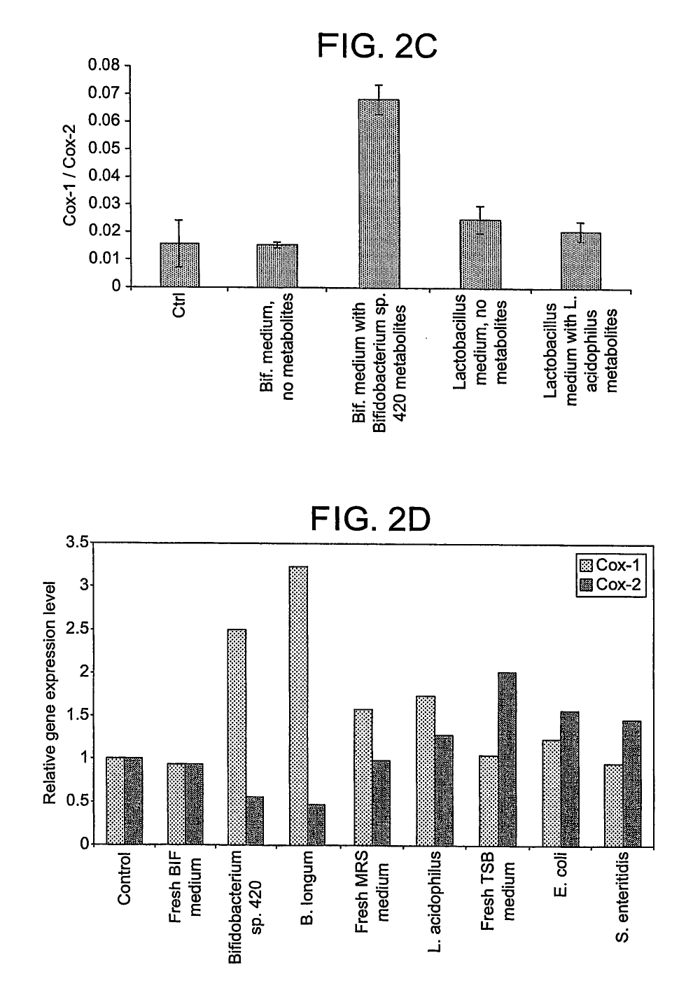

FIG. 3 shows the total damage area (mm²) observed in rats from five different treatment groups: the baseline control, indomethacin challenged, indomethacin challenged with intervention with Bifidobacterium (10⁸ daily dose of live bacteria per animal), indomethacin challenged with intervention with Bifidobacterium (10¹⁰ daily dose of live bacteria per animal), and indomethacin challenge with betaine (200 mg daily dose per animal) group. 10 rats were included in each of the treatment groups.

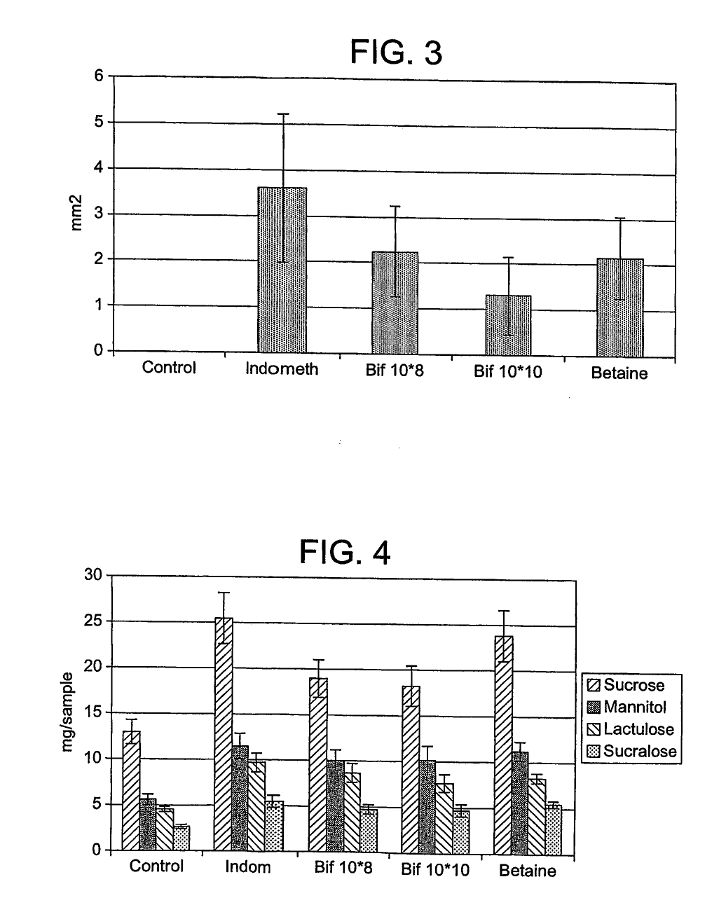

FIG. 4 shows urine concentrations of sucrose, mannitol, lactulose and sucralose in rats divided in to five different treatment groups: the baseline control, indomethacin challenged, indomethacin challenged with intervention with Bifidobacterium (10⁸ daily dose of live bacteria per animal), indomethacin challenged with intervention with Bifidobacterium (10¹⁰ daily dose of live bacteria per animal), and indomethacin challenge with betaine (200 mg daily dose per animal) group. 10 rats were included in each of the treatment groups.

### EXAMPLES

### Experiment 1

### Effect of Selected Microbial Metabolites and of Bifidobacterium sp. 420 on Cox-1/Cox-2 Expression Pattern

### Materials and Methods

The human colorectal carcinoma cell line Caco-2 was grown at 37° C. and 5% CO₂ in serum-free Dulbeccos' MEM (Gibco) supplemented with 1 mM sodium pyruvate (Gibco) and 1× non-essential amino acids (Gibco) (DMEM). When grown without added bacteria, 20 Uml⁻¹ penicillin (Gibco), 20 μgml⁻¹ streptomycin (Gibco) and 0.5 μgml⁻¹ amphotericin (Gibco) were added to the medium. Bifidobacterium sp. 420 was grown anaerobically for 48 hours at 37° C. in a BIF medium consisting of 10 gl⁻¹ tryptic digest of casein peptone; 5 gl⁻¹ meat extract; 5 gl⁻¹ yeast extract; 10 gl⁻¹ glucose; 3 gl⁻¹ K₂HPO₄; 0.1% (volume/volume) Tween 80; 1% ascorbic acid and 0.05% cysteine-HCl; pH 6.8. 2 ml of logarithmic phase culture was centrifuged at 4000 rpm for 3 minutes. The cell pellet was suspended in 1.6 ml DMEM. The production of short chain organic acids by the bacterium was tested from fresh and conditioned bifidobacterium medium using gas chromatography.

To determine the effects of various treatments on the Cox-1/Cox-2 expression pattern, approximately 500 000 Caco-2 cells/well were seeded on 24-well cell culture plates. The cells were allowed to attach for 24 hours, after which the media were replaced by fresh media (total volume 1 ml/well) containing either no other added components than the ones described above (control treatments, with and without antibiotics) or antibiotic supplemented media with 5 mM sodium butyrate; 5 mM sodium propionate; 5 mM sodium acetate or 5 mM sodium lactate. In addition, wells were prepared that contained no antibiotics but a 0.2 μm anopore membrane tissue culture insert (Nunc, Denmark) into which 100 μl of the bacterial suspension described above was added.

After 24 hour exposure, media and culture inserts were discarded, cells were lysed and RNA was extracted using Qiagen's (Germany) RNEasy Mini Kit DNA was digested using the same manufacturer's RNase free DNase. Reverse transcription was performed using the High Capacity cDNA Archive Kit (Applied Biosystems, USA) according to the instructions provided by the manufacturer. Cox-1/Cox-2 expression patterns were determined by real-time quantitative TaqMan PCR (Holland et al., 1991 Proc. Natl. Acad. Sci. USA August 15; 88(16): 7276-80; and Livak and Scmittgen, 2001 Methods December; 25(4):402-8) using the default settings of an ABIPrism 7000 Sequence Detection instrument (Applied Biosystems).

### Results and Discussion

Table 1 and FIG. 1 show the relative expression levels of Cox-1 and Cox-2 in the different treatments. According to prior art, it is known that butyrate and propionate have a lowering effect on Cox-2 expression, thus these compounds have been included as a control.

TABLE 1
Effects of selected metabolites and
a co-cultured micro-organism on cyclooxygenase 1 and 2
gene expression levels in Caco2 cells after 24 hour treatment
Expression levels are shown as fold change in relation to the control.

| Treatment | Cox-1 | Cox-2 |
|---|---|---|
| Ctrl | 1.00 | 1.00 |
| 5 mM acetate | 2.08 | 1.17 |
| 5 mM butyrate | 30.96 | 0.15 |
| 5 mM propionate | 14.70 | 0.36 |
| 5 mM lactate | 0.91 | 0.74 |
| Co-cultured Bifidobacterium sp. 420 | 2.78 | 0.15 |

As can be seen in the FIG. 1, butyrate, propionate and Bifidobacterium sp. 420 cause an increase in Cox-1/Cox-2 ratio. Particularly, they cause an increase in Cox-1 expression level. Since the products of Cox-1 enzymatic activity are protective in nature, whereas Cox-2 products are associated with inflammation and carcinogenesis (Vane, 2000 J Physiol Pharmacol December; 51(4 Pt 1):573-86 and Prescott and Fitzpatrick, 2000 Biochim Biophys Acta. March 27; 1470(2):M69-78), this kind of an effect in Cox-1/Cox-2 expression pattern is considered beneficial.

Inhibition of Cox-1 enzymatic activity can be detrimental; this is the main cause of the well-known side effects of non-steroidal anti-inflammatory drugs. It is likely that, in vivo, an equally detrimental condition is achieved in various situations where Cox-1 transcription level is decreased. In both of these situations—inhibition of Cox-1 enzymatic activity and decrease of Cox-1 transcription—an agent, such as a microorganism (or metabolite thereof) capable of modulating host cyclooxygenase expression, could be used to normalise Cox-1 expression level by increasing it so that sufficient Cox-1 enzymatic activity is achieved.

Table 2 shows the volatile fatty acid contents of fresh and filtered, conditioned bifidobacterial growth medium.

TABLE 2
Volatile fatty acid contents (in mmol/l) of fresh
medium and of bifidobacterium
conditioned by Bifidobacterium sp. 420

| Volatile fatty | Fresh medium | Conditioned |
|---|---|---|
| Acetic acid | 3.13 | 24.13 |
| Propionic acid | — | — |
| Isobutyric acid | — | — |
| Butyric acid | 0.11 | 0.16 |
| 2-methylbutyric | — | — |
| Isovaleric acid | — | — |
| Lactic acid | — | 8.57 |
| Valeric acid | 1.44 | 1.26 |

As can be seen in the table, Bifidobacterium sp. 420 produces acetate and lactate but only a minimal amount of butyrate, and no propionate. According to prior art, it is known that butyrate and propionate have a lowering effect on Cox-2 expression. In the present work it becomes evident that
1) Bifidobacterium sp. 420 has a beneficial effect on Cox-1/Cox-2 expression pattern and
2) Factors other than production of butyrate or propionate are relevant in the effect Bifidobacterium sp. 420 has on Cox-1/Cox-2 expression pattern

We conclude that the distorted Cox-1/Cox-2 expression pattern seen in many disorders, including disorders of the intestine, can be corrected without any known side-effects using a microorganism and/or a microbial suspension (for example comprising at least one metabolite of the microorganism) capable of affecting the expression pattern. By modifying the expression pattern using microorganisms or a microbial suspension thereof (for example comprising at least one metabolite of the microorganism) the status of the cells, tissues, organs or organisms can be shifted from a tumourigenic and carcinogenic state to anti-tumourigenic and anti-carcinogenic.

### Experiment 2

### Effects of Selected Microbial Strains on Intestinal Epithelial Cox-1/Cox-2 Expression Pattern

### Materials and Methods

Two bifidobacteria (Bifidobacterium sp. 420 and Bifidobacterium longum 913) were cultured as described in Experiment 1. Lactobacillus acidophilus 770, Escherichia coli (ATCC 1175) and Salmonella enteritidis were grown aerobically at 37° C. in MRS broth (Becton Dickinson, USA; lactobacillus) or in tryptic soy broth (TSB, LAB M, England; E. coli and S. enteritidis). Bacterial growth was stopped by chilling confluent cultures on ice, after which cell densities were determined by flow cytometry (FACS Calibur, Becton Dickinson). Culture supernatants were filtered through 0.22 μm sterile filter units (Mllipore, USA). The short chain organic acid contents of the supernatants and of fresh BIF, MRS and TSB media were determined using gas chromatography.

Filtered bacterial culture supernatants were diluted 1/10 in serum-free Dulbeccos' MEM (Gibco) supplemented with 1 mM sodium pyruvate (Gibco) and 1× non-essential amino acids (Gibco). The effects of soluble microbial metabolites on Cox-1/Cox-2 expression pattern were then determined using the Caco-2 cell-based exposure test described in Experiment 1. Two kinds of controls were included in the experiment: a base-level control (hereafter called control) where Caco-2 cells were only given DMEM; and three bacterial growth medium controls where caco-2 cells were given 10% unused BIF, MRS or TSB medium diluted in DMEM. The latter controls were included in the experiment to confirm that any potential changes seen in Cox-1/Cox-2 expression pattern would not be caused by some component of the bacterial growth media.

### Results and Discussion

Table 3 and FIG. 2 show the expression levels of Cox-1 and Cox-2 in Caco-2 cells exposed to metabolites produced by six different bacterial strains or to fresh bacterial culture media in relation to a control treatment.

TABLE 3
Relative Cox-1 and Cox-2 gene expression profiles in Caco-2 cells
after 22 hour exposure to 10% soluble metabolites produced by
five different microbial strains. Expression levels are given as
fold change in relation to the control treatment. For details about
the experiment, please refer to experiment 2.

| Treatment^{a} | Cox-1 | Cox-2 |
|---|---|---|
| Control | 1 | 1 |
| Fresh BIF medium | 0.93 | 0.93 |
| Bifidobecterium sp. 420 | 2.5 | 0.56 |
| B. longum | 3.23 | 0.47 |
| Fresh MRS medium | 1.58 | 0.98 |
| L. acidophilus | 1.74 | 1.28 |
| Fresh TSB medium | 1.04 | 2.02 |
| E. coli | 1.23 | 1.57 |
| S. enteritidis | 0.95 | 1.46 |

^{a}Control = Caco-2 cells maintained in serum-free Dulbecco's MEM with 1 X non-essential amino acids and 1 mM sodium pyruvate (DMEM); Fresh BIF, MRS or TSB medium = 10% fresh, unused bacterial culture media in DMEM.

Fresh bacterial culture media had little effect on Cox expression profile, although TSB medium caused an approximately 2-fold induction of Cox-2 in relation to the control treatment Within 22 hours, metabolites produced by bifidobacteria caused a 2.5 (Bifidobacterium sp. 420) and 3.2 (B. longum)-fold increase in Cox-1 expression with a simultaneous decrease (0.5 and 0.6-fold for B. sp. 420 and B. longum, respectively) in Cox-2 expression. L. acidophilus had a different effect: Cox-1 and Cox-2 expression levels, compared to the untreated control, were 1.7 and 1.3. These changes were hardly distinguishable from those caused by fresh MRS medium (1.6 and 1.0 for Cox-1 and Cox-2, respectively), for which reason it is questionable whether L. acidophilus had any effect at all. E. coli and S. enteritidis caused no significant change in Cox-1 expression either but a slight up-regulation of Cox-2 (1.6 and 1.5, respectively)—this induction, however, was less than that caused by fresh TSB medium, so based on this, the metabolites produced by these two bacterial species probably do not have a significant effect on Cox expression pattern.

Based on these results, it can be concluded that the ability to modulate host cyclooxygenase expression profile is not common to all probiotic and non-probiotic microorganisms. Only specific microorganisms are capable of producing such an effect. In this experiment, it has been clearly shown that the bifidobacteria that were tested caused an anti-tumourigenic and anti-inflammatory effect similar to that obtained using butyrate or propionate (Experiment 1). L. acidophilus and E. coli, both of which are used as probiotics, did not have such an effect. Neither did S. enteritidis, which is a pathogenic organism.

Of course, a skilled person following the teachings in the present application would have the ability to screen probiotic and non-probiotic microorganisms to identify specific microorganisms, additional to the ones specifically taught herein, capable of producing the claimed effect. In particular, the skilled person could screen microorganisms using the “Caco-2 cell-based exposure assay” taught above. Microorganisms which cause an increase in Cox-1 expression level and/or increase the Cox-1/Cox-2 ratio compared with an untreated control, may be microorganisms which can be used in accordance with the present invention.

Cyclooxygenases (Cox) 1 and 2 play important roles in gastrointestinal health; chronic overexpression of Cox-2 is associated with inflammatory and cancerous disease, whereas Cox-1 is expressed constitutively and its inhibition results in gastrointestinal malfunction. We set up a standardised cell culture-based screening assay for investigation of the effects of food components on intestinal epithelial gene expression profiles. In this model, we studied the effects of two probiotic bacterial strains (Bifidobacterium sp. 420 and Lactobacillus acidophilus) on the expression levels of the Cox genes in the enterocyte-like Caco-2 cells. Bifidobacterial metabolites shifted the Cox-1/Cox-2 ratio by increasing the amount of Cox-1 transcription 2.5-fold while simultaneously decreasing the amount of Cox-2 mRNA 0.5-fold. L. acidophilus had no effect on the amounts of Cox-1 or Cox-2. The beneficial effect of Bifidobacterium sp. 420 on cyclooxygenase expression was not mediated by butyrate, since these two bacteria did not produce butyrate. This is the first piece of evidence showing a direct relationship between a probiotic microorganism and host cyclooxygenase expression profile. The ability of a nutraceutical to induce such health-promoting transcriptional changes may provide an important criterion in the selection of novel anti-inflammatory and anticarcinogenic functional food ingredients.

### Experiment 3

### Effects of Live Bifidobacterium and Betaine on Intestinal Inflammation Caused by Nonsteroidal Anti-inflammatory Drugs in Rats

### Materials and Methods

A previously described rat model was used to study indomethacin-induced gastrointestinal damage (see Meddings and Gibbons, Gastroenterology 1998; 114:83-92). Briefly, male Wistar rats were used and ten rats were included in each treatment group. Five different treatments were included: control group with no indomethacin treatment, indomethacin control group with no intervention treatment, indomethacin with 10⁸ Bifidobacterium per rat, indomethacin with 10¹⁰ Bifidobacterium per rat, and indomethacin with betaine (200 mg/rat). A single dose of indomethacin (10 mg/kg) was given p.o. preceded by a seven-day intervention with or without Bifidobacterium or betaine. In addition, a mixture of sucrose, lactulose, mannitol and sucralose was given p.o to measure gastrointestinal (GI) permeability. Urine samples were obtained at 16 hours after the challenge with indomethacin and prior to sacrificing the animals. Mucosa of the stomach and the intestine were examined by a pathologist and tissue samples of healthy-appearing mucosa for RNA isolation were collected from stomach, proximal and distal small intestine, caecum and proximal and distal colon. RNA was isolated and expression of cyclooxygenase-1 and -2 (COX-1 and COX-2, respectively) were determined by QPCR.

### Results and Discussion

No mortality and no differences in the weight gain during the experiment were observed. A clear effect by indomethacin challenge was observed in the rat intestinal tissue. Indomethacin induced ulceration mainly to fundic stomach, but also to pyloric stomach. No other gross pathological changes were observed in the intestine (FIG. 3). Consistent with the observations of pathological lesions, the intestinal lining became permeable. A significant increase in the urine concentrations of marker sugars was observed after the challenge with indomethacin. Supplementation by Bifidobacteria resulted in reduction of pathological damage area, and especially in the numbers of rats affected by the indomethacin challenge. Betaine also exhibited a tendency to decrease the damage area caused by the challenge. Significant reduction in the permeability was observed concomitant to reduction of damage area in the groups treated with live Bifidobacteria. In contrast, the intervention with betaine did not reduce the urine sugar concentrations (FIG. 4). No differences were observed in the expression of cox-1 or cox-2 in the tissue samples obtained from healthy-appearing mucosa throughout the whole intestinal length.

Based on these results it can be concluded that the in vitro findings of reduction of inflammatory responses obtained from the Caco-2 cell model by bifidobacteria were conformed in a rat model. Not all compounds with effects on cyclooxygenase expression can reduce gastrointestinal side effects of the nonsteroid anti-inflammatory drugs. Betaine had no effect on the permeability caused by the indomethacin challenge. However, the damage area was somewhat reduced. It may be that the protective effect caused by betaine is mediated by a different mechanism to the one mediated by bifidobacteria. Lack of effect of the intervention by bifidobacteria in the expression profile of cox-1 or cox-2 in the intestinal tissue not exhibiting clear damage indicates that the effect is limited to compromised areas in the mucosa This feature widens the safe use of the tested bifidobacteria also in healthy individuals with no evident parallel challenge e.g. by nonsteroid drugs.

### Experiment 4

### Combined Effects of Live Bifidobacterium and Betaine on Intestinal Inflammation Caused by Nonsteroid Anti-inflammatory Drugs in Rats

### Materials and Methods

Male Wistar rats as described previously in Experiment 3 above are split into the following treatment groups: a control, an indomethacin challenge group as a control, and treatment groups 3-5, which receive intervention treatment by live Bifidobacterium (10¹⁰ daily dose per animal) and betaine (200 mg of daily dose per animal) either separately or in combination. Damage is determined from the sacrificed rats only.

### Results and Discussion

Preliminary investigations suggest that no mortality or differences between weight gain in the different treatment groups is observed. The clear protective effect by Bifidobacterium is repeatedly demonstrated by reduced area of damage in the gastrointestinal tract. In addition, the betaine treatment tends to decrease the damage area as observed previously. The combination of betaine with bifidobacteria exhibits a synergistic effect and damage area not significantly different from the baseline control group. The synergy between the two ingredients probably results in the different mechanism mediating the protective effect as indicated by the different effect on the permeability of the mucosa in the Experiment 2. It is concluded that combined use of betaine and certain live bifidobacteria may give additional protection against intestinal inflammatory challenges when compared with use of live bifidobacteria alone, i.e., without betaine.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. Use of a microorganism and/or a metabolite thereof in the manufacture of a medicament for use in increasing the amount of a COX-1 mRNA in a cell.

2. Use according to claim 1, wherein the microorganism and/or the metabolite thereof modifies the amount of a further cyclooxygenase mRNA in said cell.

3. Use according to claim 1, wherein the microorganism and/or the metabolite thereof increases the amount of a COX-1 mRNA in said cell, whilst simultaneously decreasing the amount of a COX-2 mRNA in said cell.

4. Use of a microorganism and/or a metabolite thereof capable of increasing at least the amount of a COX-1 mRNA in a cell, in the manufacture of a medicament for use in the prevention and/or treatment of one or more of the following: a dermatological disorder or disease; cancers of the gastrointestinal tract; inflammatory intestinal problems and diseases; trauma of intestinal mucosa; enteropathies; reco-very from surgery and skin wounds; diarrhea; nephropathies; arteriosclerosis; hypertension; liver damage; autoimmune diseases; aging; fatigue; glomerulonephritis; infectious diseases caused by pathogenic microorganisms; alopecia areata; conjunctivitis; keratitis; gastric ulcers; ischemic bowel disease; necrotizing enterocolitis; intestinal lesions; Coeliac diseases; proctitis; anemia, sarcoidosis; fibroid lung; idiopathic interstitial pneumonia chronic rheumatoid arthritis; multiple sclerosis; Alzheimer's disease; anorexia; migraine, arthritis deformans; asthma; hay fever; periodontal diseases; urogenital diseases; respiratory disorders and endotoxic shock.

5. Use of a microorganism and/or a metabolite thereof capable of increasing at least the amount of a COX-1 mRNA in a cell, in the manufacture of a medicament for use in increasing the tolerance of a subject to immunomodulating agents and/or anti-inflammatory drugs and/or increasing the tolerance of a subject to antibiotic agents.

6. Use of a microorganism and/or a metabolite thereof capable of increasing at least the amount of a COX-1 mRNA in a cell, in the manufacture of a medicament for use in the prevention and/or treatment of a side effect associated with nonsteroidal anti-inflammatory drugs.

7. Use according to claim 6 wherein the amount of a COX-1 mRNA in said cell is increased 2-fold compared with an untreated cell.

8. Use according to claim 6 wherein the microorganism is a bacterium.

9. Use according to claim 6 wherein the microorganism is from the genus Bifidobacterium.

10. Use according to claim 9 wherein the microorganism is one or more of: Bifidobacterium sp. 420, Bifidobacterium lactis, Bifidobacterium longum, Bifidobacterium breve, or Bifidobacterium animalis.

11. Use according to claim 1, wherein the microorganism and/or metabolite thereof is used in combination with i) betaine or a pharmaceutically acceptable salt thereof or a betaine replacement compound and/or ii) a nonsteroidal anti-inflammatory drug.

12. A pharmaceutical preparation comprising in combination a nonsteroidal anti-inflammatory drug and a microorganism and/or a metabolite thereof, which microorganism and/or metabolite thereof is capable of at least increasing the amount of a COX-1 mRNA in a cell.

13. A pharmaceutical preparation according to claim 12 wherein the microorganism is a bacterium.

14. A pharmaceutical preparation according to claim 12 wherein the microorganism is from the genus Bifidobacterium.

15. A pharmaceutical preparation according to claim 14 wherein the microorganism is one or more of: Bifidobacterium sp. 420, Bifidobacterium lactis, Bifidobacterium longum, Bifidobacterium breve, or Bifidobacterium aninialis.

16. A pharmaceutical preparation according to claim 12, wherein said preparation further comprises betaine or a pharmaceutically acceptable salt thereof, or a betaine replacement compound.

17. A method of treating decreased COX-1 gene expression in a subject in need of treatment, which method comprises administering to said subject an effective amount of a microorganism and/or a metabolite thereof, which microorganism and/or metabolite thereof at least increases the amount of a COX-1 mRNA in at least one cell of the subject.

18. A method of treating a disease, disorder or condition in a subject in need of treatment, which method comprises administering to said subject an effective amount of a microorganism and/or a metabolite thereof, which microorganism and/or metabolite thereof at least increases the amount of a COX-1 mRNA in at least one cell of the subject.

19. A method according to claim 18, wherein the disorder, disease or condition may be one or more of the following: a dermatological disorder or disease; cancers of the gastrointestinal tract inflammatory intestinal problems and diseases; trauma of intestinal mucosa; enteropathies; recovery from surgery and skin wounds; diarrhoea; nephropathies; arteriosclerosis; hypertension; liver damage; autoimmune diseases; aging; fatigue; glomerulonephritis; infectious diseases caused by pathogenic microorganisms; alopecia areata; conjunctivitis; keratitis; gastric ulcers; ischemic bowel disease; necrotizing enterocolitis; intestinal lesions; Coeliac diseases; proctitis; anemia; sarcoidosis; fibroid lung; idiopathic interstitial pneumonia; chronic rheumatoid arthritis; multiple sclerosis; Alzheimer's disease; anorexia; migraine, arthritis deformans; asthma; bay fever periodontal diseases; urogenital diseases; respiratory disorders and endotoxic shock.

20. A method of preventing and/or treating of reduced weight gain in livestock, preferably poultry, preferably chickens, which method comprises administering to said subject an effective amount of a microorganism and/or a metabolite thereof, which microorganism and/or metabolite thereof at least increases the amount of a COX-1 mRNA in at least one cell of the subject.

21. A method of improving the health of a subject, which method comprises administering to said subject an effective amount of a microorganism and/or metabolite thereof which microorganism and/or metabolite thereof at least increases the amount of a COX-1 mRNA in at least one cell of the subject.

22. A method of treating and/or preventing the side-effects associated with the administration of nonsteroidal anti-inflammatory drugs, which method comprises administering to the patient an effective amount of a microorganism and/or a metabolite thereof, which microorganism and/or metabolite thereof at least increases the amount of a COX-1 mRNA in at least one cell of the subject.

23. A method according to claim 17, wherein the microorganism and/or the metabolite thereof modifies the amount of a thither cyclooxygenase mRNA in said cell.

24. A method according to claim 17, wherein the microorganism and/or the metabolite thereof increases the amount of a COX-1 mRNA in said cell, whilst simultaneously decreases the amount of a COX-2 mRNA in said cell.

25. A method according to claim 17, wherein the microorganism is a bacterium.

26. A method according to claim 17, wherein the microorganism is from the genus Bifidobacterium.

27. A method according to claim 17, wherein the microorganism is one or more of: Bifidobacterium sp. 420, Bifidobacterium lactis, Bifidobacterium longum, Bifidobacterium breve, or Bifidobacterium animalis.

28. A method according to claim 15, wherein the subject is further administered with an effective amount of betaine or a pharmaceutically acceptable salt thereof or a betaine replacement compound.

29. A pharmaceutical pack comprising one or more compartments, wherein at least one compartment comprises one or more microorganism and/or metabolites thereof, which microorganism and/or metabolite thereof is capable of at least increasing the amount of a COX-1 mRNA in at least one cell of a subject and the same or a further compartment comprises one or more non-steroidal anti-inflammatory drugs.

30. A pack according to claim 29 wherein the microorganism is a bacterium.

31. A pack according to claim 29 wherein the microorganism is from the genus Bifidobacterium.

32. A pack according to claim 29, wherein the microorganism is one or more of: Bifidobacterium sp. 420, Bifidobacterium lactis, Bifidobacterium longum, Bifidobacterium breve, or Bifidobacterium animalis.

33. A pack according to claim 29, wherein at least one compartment comprises betaine or a pharmaceutically acceptable salt thereof or a betaine replacement compound.

34. A process of preparation of a pharmaceutical composition said process comprising admixing one or more microorganisms and/or metabolites thereof, which microorganism and/or metabolite thereof is capable of at least increasing the amount of a COX-1 mRNA in at least one cell of a subject, with one or more nonsteroidal anti-inflammatory drugs, and with a pharmaceutically acceptable diluent, excipient or carrier.

35. A process according to claim 34 wherein the process further comprising admixing with betaine or a pharmaceutically active salt thereof or a betaine replacement compound.

36. A pharmaceutical preparation comprising in combination a microorganism and/or a metabolite thereof and betaine or a pharmaceutically acceptable salt thereof or a betaine replacement compound, which microorganism and/or metabolite thereof is capable of at least increasing the amount of a COX-1 mRNA in a cell.

37. A pharmaceutical preparation according to claim 36 wherein the microorganism is a bacterium.

38. A pharmaceutical preparation according to claim 36 wherein the microorganism is from the genus Bifidobacterium.

39. A pharmaceutical preparation according to claim 38 wherein the microorganism is one or more of: Bifidobacterium sp. 420, Bifidobacterium lactis, Bifidobacterium longum, Bifidobacterium breve, or Bifidobacterium animalis.
